(19) United States
(12) Patent Application Publication
(10) Pub. No.: US 2007/0238695 A1
(43) Pub. Date: Oct. 11, 2007

(54) Synergistic method for the treatment of joint dysfunction using S-Adenosyl-Methionine and a patented mixture of cetylated fatty acids

(76) Inventor: Yochanan R. Bulke, Lakewood, NJ (US)

Correspondence Address: Yochanan Bulke, 170 W. Obeflin Ave, Lakewood, NJ, 08701, US

(21) Appl. No.: 11/784,023

(22) Filed: Apr. 5, 2007

Related U.S. Application Data
(60) Provisional application No. 60/744,338, filed on Apr. 5, 2006.

(51) Int. Cl. A61K 31/7076; A61K 31/22

(52) U.S. Cl. 514/47; 514/546

(57) ABSTRACT

Life Science's breakthrough SAM-e (S-Adenosyl-Methionine)/Celadrin™ (a patented mixture of cetylated fatty acids) combination is a two-pronged approach to the treatment of arthritis. The Celadrin™ helps to restabilize the balance of the catabolism and production of cartilage components, and the SAM-e actually helps regenerate cartilage that has already been destroyed. By attacking the arthritis from both angles, Life Science has created a highly potent and safe alternative for the treatment of arthritis that has none of the negative side effects that are associated with more common methods of treatment.

The present invention relates to a synergistic method for the treatment of joint dysfunction using S-Adenosyl-Methionine and a patented mixture of cetylated fatty acids. The method consists of the following ingredients:
SAMe (S-adenosyl Methionine) Celadrin™
Celadrin is comprised of the following ingredients: Esterified Fatty Acid Carbons, Malto-Dextrin, and Arabic Gum

The main components of this invention are S-Adenosyl-Methionine (SAM-e) and a patented mixture of cetylated fatty acids called Celadrin™. SAM-e enhances joint health and mobility because sulfate groups that are released in the body during the normal metabolism of SAM-e are main components of proteoglcans, the shock-absorbing components of cartilage. Studies have shown that SAM-e actually helps regenerate cartilage that has been destroyed because these sulfate groups that are released during SAM-e metabolism are incorporated into new cartilage tissue. Celadrin™ helps protect articular tissue from damage because the presence of greater amounts of fatty acids in cartilage tissue prevents cartilage from taking in arachidonic acid, which can throw off the balance of the catabolism and regeneration of certain cartilage components. If the body is breaking down cartilage components faster than it is creating new ones, an overall loss of cartilage results. The fatty acids in Celadrin™ are taken up by cartilage tissue, therebye protecting the cartilage from high amounts of arachidonic acid and maintaining the balance of catabolism and regeneration of proteoglycans.

These components work together to enhance joint health and mobility. They address the problem of cartilage degeneration from two different angles: SAM-e helps regenerate cartilage that has already been destroyed, and Celadrin™ helps to restore balance so that further cartilage never gets destroyed in the first place.

What is claimed is:

1. A synergistic method for the treatment of joint dysfunction using S-Adenosyl-Methionine and a patented mixture of cetylated fatty acids. The Celadrin™ helps to re-stabilize the balance of the catabolism and production of cartilage components by protecting chondrocytes from too much exposure to arachidonic acid and its metabolites. The SAM-e helps regenerate cartilage that has already been destroyed. Sulfate groups, a normal by-product of SAM-e metabolism, are a key component of proteoglycans—the components of cartilage that give it shock-absorbing properties. As sulfate groups are released, they are incorporated into new cartilage tissue.
